SEQUENCE LISTING (51 sequences)

SEQ ID NO: 1 - protein, 65 aa, Methanobrevibacter smithii
  REGION 1..65: note = HmtA (A5UJP0_METS3)
MELPIAPVGRILKNAGAQRVSDDAKIALTEAIEECGNEIAQKAVGFARHAGRKTVKAEDIKLAIK

SEQ ID NO: 2 - protein, 35 aa, Methanobrevibacter smithii
  REGION 1..35: note = A5UP44_METS3 (uncharacterized protein)
MSEDHNKKEHKHCPFCGHHVDEDDMICPHCGLFIP

SEQ ID NO: 3 - protein, 43 aa, Methanobrevibacter smithii
  REGION 1..43: note = A5UNQ8_METS3 (Predicted DNA-directed RNA pol II, subunit RPC10)
MYRCPRCGTEVDHKSYMENKCPKCRYRILFKNVPEVTRIIKAR

SEQ ID NO: 4 - protein, 56 aa, Methanobrevibacter smithii
  REGION 1..56: note = A5UN59_METS3 (DNA-directed RNA polymerase subunit N)
MIPIRCLSCGKPVSAYFDEYNKRLAAGEKSKDILDDLGLNRYCCRRMLISHVETWE

SEQ ID NO: 5 - protein, 69 aa, Methanobrevibacter smithii
  REGION 1..69: note = A5UMN7_METS3 (Histone)
MSEIPKAPIARIIKDTGAERVSEDAKAELAEYLEEVARDVAIEANNVAKIAKRKTIKPEDIKLAIKNLE

SEQ ID NO: 6 - protein, 275 aa, Methanobrevibacter smithii
  REGION 1..275: note = MTD_METS3 (F420-dependent MTD)
MVVKIGIIKSGNIGTSPVLDLLLDERADRPNIDVRVFGSGAKMNPEQVEDVVPKVDQFDPDFCIFISPNPGAPGPAKARELLSEKDIPAIIIGDAPGKGKKDEMDEQGLGYIIVMSDPMIGAKREWLDPTEMAIFNADILKVLAETGALRLVQNTIDGVIDGAAAGNIELPKLIITAEKAVEAAGFENPYAKAKAIAAYEMAGAVANLDMKGCFMTKGFENFIPLVAAAHEMAASAAALADEAREIEKGNDSVLRTPHMKEGNTGCKTDLISKPE

SEQ ID NO: 7 - protein, 441 aa, Methanobrevibacter smithii
  REGION 1..441: note = A5ULN2_METS3 (Methyl-coenzyme M reductase subunit beta)
MQIYNDKIDLYGQDGKLLKSEVSLDAISPLKNPAIAKMIFDIKRSCAVNLASIEKGLRTGAMGGKSVFIPGRELDLPIVENAELIADKIKRIVQVNEDDATNVSLINNGQQLLIQLPNERLRVAADYSIASLLSGSATIQSIIDTFDINMFDASTIKTAVMGAYPQTVDLAGANISALLGPPVLLEGLGYGLRNVMANHVVAITNKNTLNAAALSSIMEQTAMFETGDASGAFERMHLLTLAYQGLNADNLVFDLVKENVKGTVGTVIQSLVGRAIEDNVIKASRKMNSGFINYEPVDWALWNAYAAAGLLAAVIVNVGAARAAQAVASTVLYYNDILEYETGLPSVDFGRTMGVGVGFSFFSHSIYGGGGPGTFHGNHVVTRHSKGCAIPCASAAMCLDAGTQMFSVQRTSSLIGTVYGTIDNLRNPIINVAKSAGELKI

SEQ ID NO: 8 - protein, 443 aa, Methanobrevibacter smithii
  REGION 1..443: note = A5ULZ6_METS3 (Methyl-coenzyme M reductase subunit beta)
MAKFDDKVDLYDDRGSLVVSDVPIEALSPLRNTAIQNIVKGVKRTVAVNLEGLEKSVKTGSVGGDKSKILGRELDIDIVANAGAIAEKMKEMIQISEDDDTKVEPISGGKRLLVQVPSKRIDVAAEYSVAPLSTATSLVQAIIDVCDVSIYDANFVKAAVLGRCPQSVDYKGSNIATMLDIPQKLEGAGYALRGVKANDFAAATLKNTFQATALASIFEQTAMFEMGDAIGAYERLHLLGLAYQGMNADNMVLDLVKDNAKEGTVGSVVNGTIARAEADGVIAPQKDLTDFSIYNTDDAALWNAYAAAGAAAAVMVNIGAARAAQGIPSTLLYFNDNIEFATGLPSIDYGRAEGVAVGFSFFSHSIYGGGGPGLFNGNHVVTRHSKGFCIPCVAAAMSLDAGTQLFSPEATSGLIKEVYSQVDEFREPLKYVALAADDIKGDI

SEQ ID NO: 9 - protein, 126 aa, Homo sapiens
  REGION 1..126: note = mAb1 VH
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARAGFRGYCSGGSCYRLPYWGQGTLVTVSS

SEQ ID NO: 10 - protein, 111 aa, Homo sapiens
  REGION 1..111: note = mAb1 VL
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDVLAARSVFGGGTKLTVLG

SEQ ID NO: 11 - protein, 127 aa, Homo sapiens
  REGION 1..127: note = mAb2 VH
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLATGPHGIASAGRRYLDYWGQGTLVTVSS

SEQ ID NO: 12 - protein, 110 aa, Homo sapiens
  REGION 1..110: note = mAb2 VL
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSRGFGVFGGGTKLTVLG

SEQ ID NO: 13 - protein, 118 aa, Homo sapiens
  REGION 1..118: note = mAb3 VH
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGWGDWEMDYWGQGTLVTVSS

SEQ ID NO: 14 - protein, 111 aa, Homo sapiens
  REGION 1..111: note = mAb3 VL
QSVLTQPPSVSAAPGQKVTISCSGSSSNIGNNYVSWYQQLPGTAPXLLIYDNNKRPSGIPDRFSGSKSGTSATLGITGLQTGDEADYYCRTYDWTHSRWVFGGGTKLTVLG

SEQ ID NO: 15 - protein, 119 aa, Homo sapiens
  REGION 1..119: note = mAb4 VH
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARDFPNTEAFDIWGQGTLVTVSS

SEQ ID NO: 16 - protein, 106 aa, Homo sapiens
  REGION 1..106: note = mAb4 VL
QSALTQPASVSGSPGQSITISCTGTSSDVGGYNYVSWYQQHPGKAPKLMIYEVSNRPSGVSNRFSGSKSGNTASLTISGLQAEDEADYYCSSYDVFGGGTKLTVLG

SEQ ID NO: 17 - protein, 125 aa, Homo sapiens
  REGION 1..125: note = mAb5 VH
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARAKVGQRYSSGRYFFDYWGQGTLVTVSS

SEQ ID NO: 18 - protein, 109 aa, Homo sapiens
  REGION 1..109: note = mAb5 VL
EIVLTQSPATLSLSPGERATLSCRASQSVSSYLAWYQQKPGQAPRLLIYDASNRATGIPARFSGSGSGTDFTLTISSLEPEDFAVYYCQQGAKDNPMTFGQGTKVEIKR

SEQ ID NO: 19 - protein, 16 aa, synthetic construct
  REGION 1..16: note = scFV linker
GGGGSGGGGSGGGGST

SEQ ID NO: 20 - protein, 10 aa, Homo sapiens
  REGION 1..10: note = mAb1 H-CDR1
GYSFTSYWIG

SEQ ID NO: 21 - protein, 8 aa, Homo sapiens
  REGION 1..8: note = mAb1 H-CDR2
IYPGDSDT

SEQ ID NO: 22 - protein, 19 aa, Homo sapiens
  REGION 1..19: note = mAb1 H-CDR3
ARAGFRGYCSGGSCYRLPY

SEQ ID NO: 23 - protein, 8 aa, Homo sapiens
  REGION 1..8: note = mAb1 L-CDR1
SSNIGNNY

SEQ ID NO: 24 - skipped

SEQ ID NO: 25 - protein, 10 aa, Homo sapiens
  REGION 1..10: note = mAb1 L-CDR3
GTWDSRGFGV

SEQ ID NO: 26 - protein, 10 aa, Homo sapiens
  REGION 1..10: note = mAb2 H-CDR1
GYSFTSYWIG

SEQ ID NO: 27 - protein, 8 aa, Homo sapiens
  REGION 1..8: note = mAb2 H-CDR2
IYPGDSDT

SEQ ID NO: 28 - protein, 20 aa, Homo sapiens
  REGION 1..20: note = mAb2 H-CDR3
ARLATGPHGIASAGRRYLDY

SEQ ID NO: 29 - protein, 8 aa, Homo sapiens
  REGION 1..8: note = mAb2 L-CDR1
SSNIGNNY

SEQ ID NO: 30 - skipped

SEQ ID NO: 31 - protein, 10 aa, Homo sapiens
  REGION 1..10: note = mAb2 L-CDR3
GTWDSRGFGV

SEQ ID NO: 32 - protein, 10 aa, Homo sapiens
  REGION 1..10: note = mAb3 H-CDR1
GYTFTSYGIS

SEQ ID NO: 33 - protein, 8 aa, Homo sapiens
  REGION 1..8: note = mAb3 H-CDR2
ISAYNGNT

SEQ ID NO: 34 - protein, 11 aa, Homo sapiens
  REGION 1..11: note = mAb3 H-CDR3
ARGWGDWEMDY

SEQ ID NO: 35 - protein, 8 aa, Homo sapiens
  REGION 1..8: note = mAb3 L-CDR1
SSNIGNNY

SEQ ID NO: 36 - skipped

SEQ ID NO: 37 - protein, 11 aa, Homo sapiens
  REGION 1..11: note = mAb3 L-CDR3
RTYDWTHSRWV

SEQ ID NO: 38 - protein, 10 aa, Homo sapiens
  REGION 1..10: note = mAb4 H-CDR1
GFTFSSYSMN

SEQ ID NO: 39 - protein, 8 aa, Homo sapiens
  REGION 1..8: note = mAb4 H-CDR2
ISSSSSTI

SEQ ID NO: 40 - protein, 12 aa, Homo sapiens
  REGION 1..12: note = mAb4 H-CDR3
ARDFPNTEAFDI

SEQ ID NO: 41 - protein, 9 aa, Homo sapiens
  REGION 1..9: note = mAb4 L-CDR1
SSDVGGYNY

SEQ ID NO: 42 - skipped

SEQ ID NO: 43 - protein, 5 aa, Homo sapiens
  REGION 1..5: note = mAb4 L-CDR3
SSYDV

SEQ ID NO: 44 - protein, 10 aa, Homo sapiens
  REGION 1..10: note = mAb5 H-CDR1
GYSFTSYWIG

SEQ ID NO: 45 - protein, 8 aa, Homo sapiens
  REGION 1..8: note = mAb5 H-CDR2
IYPGDSDT

SEQ ID NO: 46 - protein, 18 aa, Homo sapiens
  REGION 1..18: note = mAb5 H-CDR3
ARAKVGQRYSSGRYFFDY

SEQ ID NO: 47 - protein, 6 aa, Homo sapiens
  REGION 1..6: note = mAb5 L-CDR1
QSVSSY

SEQ ID NO: 48 - skipped

SEQ ID NO: 49 - protein, 10 aa, Homo sapiens
  REGION 1..10: note = mAb5 L-CDR3
QQGAKDNPMT

SEQ ID NO: 50 - DNA, 21 bp, synthetic construct
  source 1..21: mol_type = other DNA
aacagaaaacccagtgaagag

SEQ ID NO: 51 - DNA, 20 bp, synthetic construct
  source 1..20: mol_type = other DNA
agtaaaggcactgaaaaacc

(19) United States
(12) Patent Application Publication
(10) Pub. No.: US 2025/0180556 A1
(43) Pub. Date: Jun. 5, 2025

(54) METHANOBREVIBACTER SMITHII ( M. SMITHII) FOR USE AS A BIOMARKER AND IN THE DIAGNOSIS AND THE TREATMENT OF DISORDERS ASSOCIATED WITH ABERRANT MICROBIOTA AND/OR ARCHAEA-DEFICIENCY

(71) Applicant: ECOLE POLYTECHNIQUE FEDERALE DE LAUSANNE (EPFL), Lausanne (CH)

(72) Inventor: Duncan-Bruce SUTHERLAND, Lonay (CH)

(21) Appl. No.: 18/845,614

(22) PCT Filed: Mar. 10, 2023
(86) PCT No.: PCT/EP2023/056151

§ 371 Date: Sep. 10, 2024

(30) Foreign Application Priority Data
Mar. 11, 2022 (EP) 22161738.4

(51) Int. Cl. G01N 33/569; A61K 35/741

(52) U.S. Cl. CPC G01N 33/56911; A61K 35/741; G01N 2333/195

(57) ABSTRACT

The present invention generally relates to the field of intestinal health, disorders associated with aberrant microbiota and/or archaea-deficiency, and early-life immune system development. The present invention more specifically relates to M. smithii and components thereof for use as a biomarker, to methods for detecting M. smithii and/or for monitoring colonization status of M. smithii, biosensors and kits for detecting M. smithii or markers thereof, and M. smithii for promoting immune system homeostasis and intestinal health and for treating disorders associated with aberrant microbiota and/or archaea-deficiency.

### TECHNICAL FIELD

The present invention generally relates to the field of intestinal health, disorders associated with aberrant microbiota and/or archaea-deficiency, and early-life immune system development. The present invention more specifically relates to M. smithii and components thereof for use as a biomarker, to the diagnosis of M. smithii and of disorders related to archaea-deficiency, to methods for detecting M. smithii and/or for monitoring colonization status of M. smithii, biosensors and kits for detecting M. smithii or markers thereof, and M. smithii for promoting immune system homeostasis and intestinal health and for treating disorders associated with aberrant microbiota (dysbiosis) and/or archaea-deficiency.

### BACKGROUND ART AND PROBLEMS SOLVED BY THE INVENTION

Humans evolved to foster advanced mutualism with intestinal bacteria. Symbiotic microorganisms colonize the human intestine during early-life in coordination with the developing immune system. It has been found more recently that microbiota composition affects the regulation of immune cells and mucosal antibody secretions that in turn promote microbiota diversification creating a symbiotic feedback loop with the host. On the other hand, aberrant microbiota formation in early-life is linked to poor immune system development and may favor the occurrence of a variety of disorders, including autoimmunity disorders.

The human intestine hosts diverse communities of fiber-fermenting bacteria. These commensals release fermentation by-products, such as acetate and butyrate, that in turn promote immunological tolerance.

Archaea, which are evolutionary distinct to bacteria, help maintain the intestinal microenvironment that supports beneficial bacteria.

M. smithii is the dominant (>90%) archaea species in humans comprising an estimated 10.24±4.58% (mean±standard deviation, SD) of the healthy adult gut microbiome (Joon Yong Kim et al, The human gut archaeome: identification of diverse haloarchaea in Korean subjects, BMC Microbiome, August 2020.

Archaea have been neglected by the scientific community, possibly because they are more difficult to identify and cultivate than bacteria. This is due to a cell wall that is more robust than the cell wall of bacteria, which makes it more difficult to extract DNA for analysis. Furthermore, the anaerobic bioprocess for cultivating archaea remains challenging.

Barnett et al, J Allergy & Clinical Immunology, 2019, found that intestinal M. stadtmanae is inversely associated with asthma in children aged 6-10 years.

Ghavami et al, Microb Pathology, 2018, find that M. smithii is inversely associated with IBD diarrhea in adults.

Coker et al, Gastroenterology, 2019, find that M. smithii is inversely associated with colorectal cancer in individuals aged 50+.

Camara et al, Scientific Reports, 2021, report that M. smithii is absent in malnourished infants, whereas Grine et al, European J of Clinical Microbiology & Inf. Diseases, 2017, report the presence of M. smithii in newborn gastric juices.

M. smithii is a dominant archaea species indigenous in the adult colon, is the main hydrogen sink enabling fermenting bacteria to thrive. Despite this key function in adult microbiota, the role of M. smithii in development of infant microbiota and its association with immune system development in early-life has not previously been investigated.

It is not known whether M. smithii colonizes the intestine in coordination with the immune system in an age-dependent manner; and, moreover, whether timely M. smithii colonization correlates with normal immune system development.

Infant gut colonization plays an important role in early-life immune system development (Tamburini S, Shen N, Wu H, Clemente J, Microbiome in early life: implications for health outcomes, 2016 Nature Medicine). Therefore, there is a need for pediatricians to monitor colonization status associated with normal immune system development.

Despite massive research efforts, the monitoring of microbiome status has remained complex, costly and often ambiguous since there are thousands of bacterial strains and there is a high degree of epidemiological variation between different populations.

The present inventor finds that M. smithii archaea is a keystone species of the human microbiota that serves as the hydrogen sink for fiber-associated microbiota. Fiber-associated microbiota play a known role in immune system regulation in infants and in preventing asthma and allergy (Maslowski K. et al, Regulation of inflammatory responses by gut microbiota and chemoattractant receptor GPR43, 2009, Nature).

In view of the prior art, there is a need to understand better the role of M. smithii and intestinal health, immune system homeostasis, and disorders associated with aberrant microbiota, archaea-deficiency, and M. smithii deficiency. There is also a need for treating and/or preventing such disorders.

There is a need for a test that allows monitoring gut colonization status associated with normal immune system development. A test capable of more easily measuring archaea levels in stool samples is needed to more easily monitor the role of M. smithii archaea in a variety of health contexts notably pediatrics.

Further objectives and problems addressed by the present invention will become apparent from the description of the aspects and embodiments of the invention herein below.

### SUMMARY OF THE INVENTION

The present invention provides the use of M. smithii and markers for detecting M. smithii as biomarkers. The present invention also provides methods for treating and/or preventing one or more conditions as disclosed in this specification.

In an aspect, the invention provides a marker comprising an amino acid sequence of any one of according to any one of SEQ ID NO: 1 to 8, or comprising an amino acid sequence having at least 80% sequence identity with any one selected from SEQ ID NO: 1 to 8, for detecting and/or determining M. smithii. In an aspect, the invention provides HmtA as a preferred marker of M. smithii.

In an aspect, the invention provides the use of a protein comprising the amino acid sequence of any one of SEQ ID NO: 1-8, or an amino acid sequence having at least 80% sequence identity with any one selected from SEQ ID NO: 1 to 8, as a marker for M. smithii.

In an aspect, the invention provides a binding molecule, for example a probe or an antibody or fragment thereof, for detecting, assessing, monitoring and/or determining (e.g. quantitatively and/or semi-quantitatively) M. smithii. Preferably, the binding molecule is specific to M. smithii.

In an aspect, the invention provides a binding protein that specifically binds to a marker protein of M. smithii, wherein said marker protein is selected from the proteins comprising an amino acid sequence of any one of SEQ ID NO: 1-8, or an amino acid sequence having at least 80% sequence identity with any one selected from SEQ ID NO: 1 to 8.

In further aspects, the invention provides a kit or assay, for example a diagnostic kit, for detecting, determining and/or assessing M. smithii.

In an aspect, the diagnostic kit is for monitoring and/or assessing one or more selected from the group consisting of: immune system homeostasis, intestinal immune homeostasis, and intestinal health, preferably in a human newborn, infant and/or a toddler up to 6, 5 or 4 years.

In an aspect, the diagnostic kit is for monitoring and/or assessing one or more selected from the group consisting of: gut colonization status, mucosal immune barrier function, intestinal immune system status, and intestinal adaptive immune system status, preferably in a human newborn, infant and/or a toddler up to 6, 5 or 4 years.

In an aspect, the diagnostic kit is for diagnosing one or more selected from the group consisting of: an aberrant microbiota, dysbiosis, archaea-deficiency, and M. smithii deficiency in a human newborn, infant or a toddler up to 6, 5 or, preferably, up to 4 years.

The diagnostic kit of the invention preferably comprises a binding molecule for detecting and/or determining or approximating a quantity of M. smithii in a sample taken from the individual.

In an aspect, the invention provides the use of M. smithii or of a marker thereof for assessing the risk that an individual suffers from or will develop one or more selected from the group consisting of: colic, allergy, asthma, IBD (Inflammatory Bowel Disease) and diarrhoea, wherein said human individual is selected from the group of newborns, infants, and toddlers up to 6, 5 or up to 4 years.

In an aspect, the invention provides the use of M. smithii for monitoring and/or assessing immune system homeostasis, intestinal immune homeostasis, and intestinal health, in a human newborn, infant or a toddler up to 6, 5 or up to 4 years.

In an aspect, the invention provides the use of M. smithii for promoting one or more selected from the group consisting of: immune system homeostasis, intestinal immune homeostasis, intestinal health, gut colonization status, mucosal immune barrier function, intestinal immune system status, intestinal adaptive immune system status, modulating IgA homeostasis and modulating T cell homeostasis.

In an aspect, the invention provides the use of M. smithii for treating and/or preventing one or more selected from the group consisting of: dysbiosis, an aberrant microbiota, archaea-deficiency, and M. smithii deficiency.

In an aspect, the invention provides the use of M. smithii for treating and/or preventing one or more selected from the group consisting of: colic, allergy, asthma, colon cancer, IBD and diarrhoea.

In an aspect, the invention provides the use of M. smithii for regulating microbiota and/or immune system development.

In preferred embodiments, the invention is destined to individuals, preferably human individuals, early in life, for example newborns, infants and toddlers, in particular toddlers up to 6, 5 or up to 4 years.

The invention also provides the uses of the invention destined to individuals, preferably human individuals, being 50 years old or older. For example, M. smithii, the markers and/or binding molecules of the invention are used to assess a risk that the individual will develop colon cancer. Furthermore, formulations comprising M. smithii may be used for preventing and/or treating colon cancer. In some embodiments, M. smithii is used for promoting one or more selected from immune system homeostasis, intestinal immune homeostasis, intestinal health and the like in a patient suffering from colon cancer.

In some aspects, the invention provides methods for detecting, determining, assessing and/or monitoring M. smithii, for example by detecting and/or assessing a marker of M. smithii. An exemplary marker is HmtA.

In an aspect, the invention provides a method for monitoring and/or assessing one or more selected from the group consisting of: immune system homeostasis, intestinal immune homeostasis, and/or intestinal health, in a human individual.

In an aspect, the invention provides a method for diagnosing one or more selected from the group consisting of: an aberrant microbiota, dysbiosis, archaea-deficiency, and M. smithii deficiency, in a human individual.

The methods of the invention preferably comprise: determining whether M. smithii is present in a sample collected from the human; and if so, determining or estimating a quantity of M. smithii in the sample, and, finding insufficient immune system homeostasis, intestinal immune homeostasis, and/or intestinal health if M. smithii is absent or is present at a quantity that is below a threshold level, and, respectively, diagnosing an aberrant microbiota, dysbiosis, archaea-deficiency, and/or M. smithii deficiency if M. smithii is absent or is present at a quantity that is below a threshold level.

In some aspects, the invention concerns treating and/or preventing one or more selected from the group consisting of: colic, allergy, asthma, colon cancer, IBD, diarrhoea, aberrant microbiota, archaea-deficiency, and M. smithii deficiency, and/or, promoting one or more selected from the group consisting of: immune system homeostasis, intestinal immune homeostasis, intestinal health, gut colonization, mucosal immune barrier function, the intestinal immune system, and the intestinal adaptive immune system, the method comprising administering, to an individual in need thereof, a composition or formulation comprising M. smithii.

Further aspects and preferred embodiments of the invention are defined herein below and in the appended claims. Further features and advantages of the invention will become apparent to the skilled person from the description of the preferred embodiments given below.

### BRIEF DESCRIPTION OF THE DRAWINGS

FIG. 1A shows age-dependent increase of M. smithii biomarker in healthy infant cohort. Microbial cells were gated using forward and side scatter and then the percentage IgA+ and F420-populations and IgA+F420+ populations determined using a quadrant gate. The respective ratio of each population was plotted. Samples with M. smithii biomarker percentage over 3.8% were marked with a circle as “M. smithii high” and those below were marked with a rhombus shape as “M. smithii low”.

FIG. 1B shows age-dependent increase of M. smithii biomarker in healthy infant cohort. The percentage of cells positive for the M. smithii biomarker F420 from each sample were measured and then the average of the samples for each age category was calculated and plotted (SD and statistics not shown).

FIG. 2A shows M. smithii relative abundance positive correlation with microbiota sIgA coating in infants. The percentage of cells positive for the M. smithii biomarker were plotted (x axis) against the percentage of IgA coated microbial cells (y axis) and a logarithmic trend line was calculated by excel formula to determine the correlation between M. smithii level and percentage of IgA coated microbiota cells.

FIG. 2B shows M. smithii relative abundance positive correlation with microbiota sIgA coating in infants. The graph compares percentage IgA coated microbiota cells between “M. smithii high” versus “M. smithii low” from age group18-24 months.

FIGS. 3A-3E shows the binding of five scFv fragments based on five monoclonal antibodies mAb1 to mAb5 to HmtA according to embodiments of the invention. Binding of the scFvs was revealed in an ELISA setting as described in the examples.

FIG. 4 shows M. smithii colonization in gnotobiotic mice after oral administration of viable M. smithii, determined by flow cytometry.

FIG. 5 shows M. smithii colonization in gnotobiotic mice after oral administration of viable M. smithii, determined by PCR.

FIG. 6 shows the mean relative abundance, determined on the basis of 16S rRNA sequencing, of fecal microbiome composition from OligoMM12 mice treated with M. smithii versus non-treated.

Hereinafter, preferred embodiments of the device of the invention are described, in order to illustrate the invention, without any intention to limit the scope of the present invention.

### DETAILED DESCRIPTION OF THE PREFERRED EMBODIMENTS

The present invention relates to M. smithii and markers allowing detection of M. smithii in a sample, in particular a sample taken from a human or animal individual. M. smithii and markers thereof may be used for diagnostic and prognostic purposes and as a biomarker, based on the role of M. smithii in immune system homeostasis and intestinal health, amongst others. The invention also encompasses prophylactic, therapeutic and other methods comprising the administration of M. smithii, in the form of viable M. smithii or inactivated M. smithii, for example.

The present invention also relates to binding molecules, for detecting M. smithii, for example by detecting the binding of the binding molecule to a marker of M. smithii. The invention also comprises using a binding molecule that is specific to M. smithii in the uses, methods and kits of the invention.

The term “marker”, for the purpose of the present specification, refers to a molecule, generally a protein or a nucleic acid molecule, which is produced by M. smithii and which allows detecting M. smithii in a sample, preferably in a specific manner. The specificity is preferably such that the “marker” allows discriminating between M. smithii and other microorganisms of the gut microbiota, preferably non-archaea microorganisms, such as gram-positive and/or gram-negative bacteria. More preferably, the “marker” also allows discriminating between M. smithii and other archaea, such as M. boviskoreani and M. stadtmonae, for example. In other words, the “marker” is preferably a molecule the detection of which allows to conclude, preferably with a high certainty, that M. smithii is present in a sample.

For the purpose of the present specification, the term “biomarker” refers generally to the use of M. smithii and markers thereof for making predictions about the health status and/or risks associated with a deficiency of M. smithii in an individual. In accordance with an embodiment, M. smithii and markers thereof are used as a biomarker.

The present invention is partially based on the surprising finding that the expansion of M. smithii in infant microbiota appears to be age-dependent and coordinated with immune system development. In various embodiments, the invention relates to assessing, determining and/or monitoring M. smithii in one or more selected from newborns, infants, and toddlers up to 6 years, preferably up to 5 years, for example up to 4 years.

Furthermore, it is known that archaea deficiency, in particular M. smithii deficiency, is associated with certain health risks in adults, for example in human individuals being 50 or older. In some embodiments, the present invention encompasses assessing, determining and/or monitoring M. smithii in adults, in particular in human individuals having 50 years or more.

For assessing, determining and/or detecting M. smithii, a sample of an individual is preferably taken. The sample may be a previously taken sample. The sample may be selected from a sample of stool, oral saliva, vaginal mucosa (pre-birth), breast milk or colostrum.

Preferably the sample is a stool sample. The stool sample is preferably treated as appropriate so as to allow for the interaction of the marker with the binding molecule and detection of the interaction. Preferably, the sample is treated so as to allow conclusions about the concentration of M. smithii in the sample, for example based on the sample characteristics, such as weight, volume, and the like.

M. smithii, or a marker thereof, may be determined qualitatively, for example finding whether or not M. smithii is present in a sample. In preferred embodiments, M. smithii is determined semi-quantitatively or quantitatively, so as to preferably allow the assessment of the concentration as described above. Preferably, in some embodiments, the binding molecule is used for determining M. smithii qualitatively, semi-quantitatively and/or quantitatively. For example, a level of the marker, such as HmtA or any other marker, is assessed semi-quantitatively or quantitatively.

For monitoring M. smithii, M. smithii is preferably determined, as described above, repeatedly. For example, M. smithii may be determined twice, three time or more, at different moments in time. For example, M. smithii is determined once a year or more often, once every two years or more often. For example, M. smithii is determined once, twice, three times or more per year. In some embodiments, M. smithii is determined and/or assessed every two months or every month, for example.

The invention provides markers that are suitable for detecting M. smithii. The markers are preferably selected from proteins and nucleic acid molecules, such as polynucleotides. Nucleic acid molecules may be selected from DNA and RNA molecules, for example. Preferably, the marker only occurs, in the particular form, for example with the particular amino acid or nucleotide sequence, only in M. smithii, or possibly in other microorganisms that do not occur in the human and/or animal microbiota.

The present inventors have analysed the proteome of M. smithii, and have identified several proteins that may be used as markers. Table 1 below lists eight different proteins, which may be used as markers for the purpose of the present invention. F420+ (SEQ ID NO: 6), a protein that has been previously reported as a marker for M. smithii, may also be used. In some embodiments, F420+ (SEQ ID NO: 6) is excluded from the markers, kits, uses and methods of the invention.

In a preferred embodiment, the marker is selected from proteins comprising an amino acid sequence according to any one of SEQ ID NO: 1 to 8, or comprising an amino acid sequence having at least 80% sequence identity, preferably at least 90% identity, and most preferably at least 95%, 97%, 98% or 99% sequence identity with any one of SEQ ID NO: 1-8. Details regarding sequence identity are provided elsewhere in this specification, in particular further below, before the example section.

Preferably, with proteins having amino acid sequences that do not have 100% identity with SEQ ID NO: 1-8, including modified proteins linked to other molecules, peptides and/or proteins, the marker is still specific for M. smithii.

In an embodiment, the marker is selected from proteins listed in Table 1 below. The sequences of these markers are given in Table 2 further below.

TABLE 1
Marker proteins for detecting M. smithii

| | | | | SEQ |
|---|---|---|---|---|
| | | Gene | Length | ID |
| Entry name | Protein names | names | (aa) | NO: |
| A5UJP0_METS3 | Histone | Msm_0213 | 62 | 1 |
| A5UP44_METS3 | Uncharacterized protein | Msm_1767 | 35 | 2 |
| A5UNQ8_METS3 | Predicted DNA-directed RNA pol | Msm_1631 | 43 | 3 |
| | II, subunit RPC10 | | | |
| | DNA-directed RNA pol subunit N | Msm_1432 | 56 | 4 |
| A5UN59_METS3 | (EC 2.7.7.6) | | | |
| A5UMN7_METS3 | Histone | Msm_1260 | 69 | 5 |
| MTD_METS3 | F420-dependent MTD (EC | Msm_1204 | 275 | 6 |
| | 1.5.98.1) | | | |
| A5ULN2_METS3 | Methyl-coenzyme M reductase | Msm_0905 | 441 | 7 |
| | subunit beta (EC 2.8.4.1) | | | |
| | Methyl-coenzyme M reductase | Msm_1019 | 443 | 8 |
| A5ULZ6_METS3 | subunit beta (EC 2.8.4.1) | | | |

It is noted that the marker protein may naturally occur in association with other molecules, for example may be fused or otherwise covalently bound to other peptides, proteins, sugars, lipids, and other molecules. Furthermore, the marker may be produced artificially. For this purpose, the marker may be purposefully fused to other peptides, proteins, sugars, lipids, and other molecules, which may be useful for various purposes, such as producing, detecting, and isolating the marker.

In a preferred embodiment, the marker comprises an isolated protein.

In an embodiment, M. smithii or the marker thereof may be used for monitoring and/or assessing one or more selected from: immune system homeostasis, intestinal immune homeostasis, intestinal health, gut colonization status, mucosal immune barrier function, intestinal immune system status, and intestinal adaptive immune system status.

In an embodiment, M. smithii or the marker thereof may be used for assessing the risk that an individual suffers from or will develop one or more selected from the group consisting of: colic, allergy, asthma, colon cancer, IBD and diarrhoea. Preferably, said individual is human newborn, infant, and/or a toddler up to 6, 5 or 4 years.

In an embodiment, M. smithii or the marker thereof may be used for diagnosing one or more selected from the group consisting of: dysbiosis, an aberrant microbiota, archaea-deficiency, and M. smithii deficiency.

In particular, the marker may be used in methods, including diagnostic or prognostic methods, risk assessment methods and the like, but also in biosensors and/or diagnostic kits.

In an embodiment, the marker for M. smithii is selected from HmtA and from F420+. In an embodiment, the invention provides the use of HmtA for detecting M. smithii in a sample.

In a preferred embodiment, the marker is or comprises HmtA. HmtA is a protein comprising or essentially consisting of the amino acid sequence of SEQ ID NO: 1 or a protein having at least 80% sequence identity with SEQ ID NO: 1. SEQ ID NO: 1 is a histone encoded by gene Msm_0213, having 65 amino acids found in M. smithii.

Preferably, said HmtA is present in the intestine of an individual or in the faeces of an individual.

Preferably, HmtA is detected in a sample taken from the intestines of an individual or in a sample of faeces of said individual.

In an embodiment, HmtA is used as a biomarker.

In an embodiment, HmtA is used as a biomarker for one or more selected from the group consisting of: immune system homeostasis, intestinal immune homeostasis, and intestinal health.

In an embodiment, HmtA is used for assessing the risk for developing one or more selected from the group consisting of: colic, allergy, asthma, colon cancer, IBD and diarrhoea.

In an embodiment, HmtA is used for assessing the risk for a human individual to develop one or more selected from the group consisting of: colic, allergy, asthma, IBD and diarrhoea, wherein said human individual is selected from newborns, infants, and toddlers of up to 6, 5 or 4 years.

In an embodiment, the invention provides the use of HmtA for assessing the risk of a human individual having an age of 50 years or higher of suffering or developing colon cancer.

In an embodiment, the invention provides the use of HmtA for detecting M. smithii in a sample.

In an embodiment, the invention comprises using a binding molecule for:
determining a presence or absence of M. smithii in a sample collected from the individual, and/or
determining or approximating a quantity of M. smithii in a sample taken from the individual.

The expression “approximating a quantity of M. smithii” is intended to encompass semi-quantitative assessment of M. smithii and/or of the marker thereof.

In an embodiment, said binding molecule comprises one or more selected from:
an oligo- or polynucleotide;
a binding protein, preferably an immunoglobulin superfamily (IgSF) protein or fragment thereof, preferably an antibody or fragment thereof.

If the binding molecule is an oligo- or polynucleotide, it is preferably single-stranded. In an embodiment, the binding molecule is probe, preferably an oligo- or polynucleotide probe.

If the marker comprises a protein, such as HmtA, it is preferably detected by way of a binding protein. The binding protein is preferably selected from binding proteins of the immunoglobulin superfamily (IgSF) or fragments of such proteins. In some embodiments, the binding protein comprises one or several fragments of one or more IgSF proteins. IgSF proteins may be selected from antibodies, TCRs (T cell receptors), BCRs (B cell receptors), CARs (chimeric antigen receptors), or a fragment of any one of the aforementioned. In a preferred embodiment, the binding protein is or comprises an antibody or a fragment thereof.

Fragments of binding proteins may be selected, for example, from polypeptides comprising one or more selected from the group consisting of: an Fc fragment (crystallizable fragment), an Fab fragment (antigen binding fragment), an Fv fragment (variable fragment), a scFv (single chain variable fragment), an Ab heavy chain, an Ab light chain, a VH domain (heavy chain variable region), a VL domain (light chain variable region), a CH (heavy chain constant region), a CL (light chain constant region). Exemplary Fab fragments encompass Fab, Fab′, F(ab′) 2 fragments.

In an embodiment, the binding protein is or comprises a human or humanized IgSF protein or fragment thereof, for example a human or humanized antibody or fragment thereof.

In a preferred embodiment, the binding protein binds to the marker and is preferably specific to the marker. In a preferred embodiment, the binding proteins binds to HmtA and is preferably specific to HmtA.

As used herein, the term “specifically binds” shall be taken to mean that the binding interaction between the binding molecule (e.g. Probe, Ab, TCR, BCR, CAR, or fragment thereof) and the marker, such as HmtA or a or a nucleic acid molecule, is dependent on the presence of an antigenic determinant or epitope of the marker protein bound by the binding protein, or of a specific nucleotide sequence in case of a nucleic acid molecule. Accordingly, the binding molecule preferentially binds or recognizes an antigenic determinant or nucleotide sequence of the marker molecule even when present in a mixture of other molecules. In one example, the binding molecule reacts or associates more frequently, more rapidly, with greater duration and/or with greater affinity with one or several particular markers, or a cell expressing said particular markers, than it does with alternative antigens, cells or nucleic acid molecules. It is also understood by reading this definition that, for example, a binding molecule that specifically binds to one or more particular markers may or may not specifically bind to a second antigen or nucleic acid molecule. As such, “specific binding” does not necessarily require exclusive binding or non-detectable binding to another molecule. Generally, reference herein to binding means specific binding, and each term shall be understood to provide explicit support for the other term. Methods for determining specific binding will be apparent to the skilled person. For example, the binding molecule of the disclosure is incubated with a particular marker, such as HmtA, or a cell expressing said particular marker, or a mutant form thereof, or an unrelated antigen or nucleic acid molecule. Binding of the binding molecule to said particular marker, to a mutant form thereof, or to the unrelated molecule is then determined and a binding molecule that binds as set out above to said particular marker rather than to the mutant, the unrelated molecule is considered to specifically bind to said particular marker.

The “binding” of the binding molecule of the invention to a marker, such as HmtA, is preferably non-covalent binding. The binding is preferably specific binding. The molecular forces involved in the binding molecule-marker binding are preferably selected from one or more from the group consisting of electrostatic forces, hydrogen bonds, hydrophobic interactions, and van der Waals forces.

In a preferred embodiment, the binding molecule is a binding protein, preferably an IgSF protein or fragment thereof. The binding protein preferably binds specifically to a marker of M. smithii.

In an embodiment, said binding protein specifically binds to a protein comprising an amino acid sequence that has at least 90% sequence identity with any one of SEQ ID NO: 1-8.

The present invention provides monoclonal antibodies that specifically bind to HmtA. In an embodiment, the binding proteins comprises one of these antibodies or fragments thereof, for example VH, VL and or one or mor CDRs of any one of these antibodies. Such fragments are listed in Table 2 below. In an embodiment, the binding protein comprises an amino acid sequence of at least one selected from SEQ ID NO: 9-49.

In an embodiment, the binding protein comprises a VH region comprising an amino acid sequence selected from SEQ ID NOs: 9, 11, 13, 15, 17, or an amino acid sequence having at least 80%, preferably at least 90%, more preferably at least 95%, 97%, 98% or 99% sequence identity, preferably at least 95% sequence identity with any one of SEQ ID NOs: 9, 11, 13, 15, 17.

In an embodiment, the binding protein comprises a VL region comprising an amino acid sequence selected from SEQ ID NOs: 10, 12, 14, 16, and 18, or an amino acid sequence having at least 80%, preferably at least 90%, more preferably at least 95%, 97%, 98% or 99% sequence identity, preferably at least 95% sequence identity with any one of SEQ ID NOs: 10, 12, 14, 16, and 18.

In an embodiment, the binding protein comprises one, two, three, preferably up to six CDR regions having a sequence selected from any one of SEQ ID NOs: 20-49, or a sequence having at least 80%, preferably at least 90%, more preferably at least 95%, 97%, 98% or 99% sequence identity with any one of SEQ ID NO: 20-49.

In an embodiment, the binding protein comprises H-CDR1, H-CDR2, and H-CDR3 amino acid sequences; and/or L-CDR1, L-CDR2 and L-CDR3 amino acid sequences selected from the group of:
a. SEQ ID NO: 20-22 and/or SEQ ID NO: 23-25,
b. SEQ ID NO: 26-28 and/or SEQ ID NO: 29-31,
c. SEQ ID NO: 32-34 and/or SEQ ID NO: 35-37,
d. SEQ ID NO: 38-40 and/or SEQ ID NO: 41-43, and
e. SEQ ID NO: 44-46 and/or SEQ ID NO: 47-49,
   or sequences having, independently, at least 80% sequence identity with any one of these sequences. Further preferred sequence identity percentages as indicated above also apply in this and other embodiments.

Preferably, the binding protein is an artificial protein or is a natural protein that has been modified so as to be different from molecules occurring in nature. Preferably, the binding protein is conjugated to one or more tags (gold, latex, fluorophore, peptide tags), for enabling one or more selected from production, isolation, manipulation, of the binding protein, and/or for allowing a read-out in a test kit comprising the binding protein.

In a preferred embodiment, the binding protein is or comprises a scFv protein, in which a VH region is fused with a VL region via a suitable linker, which is preferably an artificial linker. The linker preferably comprises from 5 to 40, preferably 10-25 amino acids. Preferably, the linker is sufficiently flexible to provide a functional scFv having the appropriate binding properties. Preferably, the linker comprises glycine for flexibility and preferably serine and/or threonine moieties, for example for solubility reasons. An exemplary linker is given in SEQ ID NO: 19.

For example, an scFv may comprise VH-VL pairs of SEQ ID NO: 9 and 10; 11 and 12; 13 and 14; 15 and 16; 17 and 18. The variable domains may be linked as appropriate, for example in an N-VH-VL-C manner or N-VL-VH-C manner, wherein N and C indicate the corresponding terminus of the fusion protein.

The binding molecule, for example the binding protein, may be used for detecting, determining and/or assessing M. smithii, for example for detecting a marker of M. smithii. The binding protein may be used for determining and/or assessing the presence of M. smithii qualitatively, semi-quantitatively or quantitatively.

In an embodiment, the binding molecule, for example the binding protein, is used for assessing one or more selected from: immune system homeostasis, intestinal immune homeostasis, intestinal health, gut colonization status, mucosal immune barrier function, intestinal immune system status, intestinal adaptive immune system status. Preferably, the binding protein is specific to HmtA, and more preferably is a binding protein according to an embodiment described in this specification.

In an embodiment, the binding molecule, for example the binding protein, is used for assessing the risk that an individual suffers from or will develop one or more selected from the group consisting of: colic, allergy, asthma, colon cancer, IBD and diarrhoea. Preferably, the binding protein is specific to HmtA and more preferably is a binding protein according to an embodiment described in this specification.

Assessing the risk that an individual will develop a particular disorder is an embodiment of the prognostic use of the M. smithii, the marker thereof, and/or of the molecule binding to the marker. Preferably, the binding molecule is a binding protein that is specific to HmtA and more preferably is a binding protein according to an embodiment described in this specification.

In an embodiment, the binding molecule, for example the binding protein, is used for diagnosing one or more selected from the group consisting of: dysbiosis, an aberrant microbiota, archaea-deficiency, and M. smithii deficiency. Preferably, the binding protein is specific to HmtA, and more preferably is a binding protein according to an embodiment described in this specification.

In an embodiment, the binding molecule, for example the binding protein, is used for assessing the risk that a human individual suffers from or will develop one or more selected from the group consisting of: colic, allergy, asthma, IBD and diarrhoea, wherein said human individual is selected from the group consisting of: newborns, infants, and toddlers up to 6, 5 or, preferably, 4 years. Preferably, the binding molecule, for example the binding protein, is specific to a marker, preferably to a marker disclosed in this specification, e.g. in Table 1, most preferably to HmtA. Preferably, the binding molecule is a binding protein according to an embodiment described in this specification, more preferably a binding protein that is specific to HmtA.

In an embodiment, the binding molecule, for example the binding protein, is used for assessing the risk that an individual suffers from or will develop colon cancer, wherein the individual is 50 years old or older and wherein the binding protein is specific to a marker of M. smithii, preferably a marker selected from the markers listed in Table 1. Preferably, the marker is HmtA. Preferably, the binding molecule is a binding protein according to an embodiment described in this specification, for example a binding protein that specifically binds to HmtA.

The present invention provides test kits, including diagnostic tests, and biosensors, for assessing and/or determining M. smithii, preferably qualitatively, semi-quantitatively or quantitatively.

The test kit may be in the form of binding-molecule, for example binding-protein based test, such as an antibody-based test. Such tests include rapid tests, rapid diagnostic tests that directly detects the presence or absence of an antigen, in particular the marker. Preferably, the rapid test gives a result within 5 minutes to 1 hour.

In some embodiments the test is a lateral flow test.

Preferably, the test comprises at least a housing, wherein detection agents, reactants, such as a binding molecule, such as a binding protein as disclosed herein, are provided at appropriate locations inside the housing, and/or in separate recipients, such as tubes, and the like.

In some kit applications, it may be advantageous to apply cell lysis solution/s and buffers to prepare samples in order to increase the detection sensitivity of the biomarker. One or more lysis solutions and/or buffers may be present in the test kit.

The test preferably comprises a sample recipient or well, where the sample, e.g. stool sample, or prepared from a stool sample, is added. Preferably, a determined amount, volume or weight of the sample is added. The recipient or well is preferably provided in the housing.

Preferably, the test comprises the binding molecule in accordance with the invention, preferably a binding protein, such as an antibody or fragment thereof that specifically binds to HmtA. The binding protein may be immobilized, or may be provided to be able to diffuse or flow, generally upon exposure to a sample.

Preferably, the test further comprises one or more further binding molecule, for example further binding proteins, for the purpose of providing a negative control and/or for immobilizing the complex of the analyte (marker) and the binding molecule, for example the binding protein, if the marker is present, in the case of a positive test outcome.

Generally, the test comprises a substrate, preferably an artificial substrate, such as a membrane, e.g. a nitrocellulose membrane, where binding molecules, such as binding proteins are deposited, generally to allow a read-out at pre-determined locations of the substrate. The substrate is preferably provided, for example supported or fixed, inside the housing.

The test preferably comprises one or more recipients, such as Eppendorf tubes and the like, where additional reactants that are required for using the test are provided.

The test kit may be used for assessing, determining and/or monitoring M. smithii. The test may also be used for one or more of the diagnostic and/or prognostic purposes disclosed in this specification.

The kits and methods of the invention may result in a negative outcome if M. smithii is absent or is present at a quantity that considered to be below a given level. This level may be considered to be a threshold level for distinguishing a healthy from an unhealthy and/or insufficient M. smithii colonization status.

In an embodiment, the threshold level is defined by a reduction of 90% or more in the relative abundance of M. smithii in stool samples compared to the mean of M. smithii abundance in the same age category, as determined for healthy individuals. In other words, the threshold level may be defined as an M. smithii count reduced by a factor of 10 (−1 log 10) compared to the average in the age group of the individual to be assessed.

If M. smithii is assessed by way of DNA analysis, infants with M. smithii deficiency have 1 log less M. smithii specific DNA relative to total bacteria DNA in stool samples compared to the mean of healthy infants from the same age category. If viable bacteria are assessed, infants with M. smithii deficiency have 1 log 10 less M. smithii viable cells (per gram stool) relative to total viable bacteria cells in stool samples compared to the mean of healthy infants from the same age category.

In a preferred embodiment, the binding molecule of the present invention is used for quantitively and/or semi-quantitatively assessing and/or determining M. smithii colonization status.

The age categories are preferably defined by the following periods, each of which defines an own age category: 6-12 months, 12-18 months, 18-24 months, 24-30 months, 30-36 months, 3-4 years, 4-5 years, 5-6 years.

The microbiota density (i.e. number of viable microbial cells per gram of stool) can be estimated using anaerobic culturing methods based on number of colony-forming units (CFU) per gram of stool. The microbiota density is approximately in the range of 10¹¹-10¹² cells per gram stool.

The relative abundance of M. smithii (e.g. determined by flow cytometry or qPCR) can be used to calculate the absolute amount of M. smithii cells per gram of stool when the microbiota density is known.

For comparing a value for an individual to be assessed with the mean value of the corresponding age category, M. smithii is preferably determined with the same methods, to ensure that a meaningful comparison is possible.

In other embodiments, an unhealthy and insufficient M. smithii colonization status resulting in a negative diagnostic assessment and/or test outcome is found if M. smithii represents 5% or less, preferably 3%, 1.5% or 1% or less of the gut microbiome. These percentages may refer to either one or both selected from viable cells (in comparison with CFUs of other bacteria) and biomass (in comparison with the biomass of other bacteria of the gut microbiome). In accordance with this embodiment, a comparison with the mean value of M. smithii in healthy individuals of the corresponding age group is not necessary, and the threshold level is determined with reference to the individual's own microbiome.

The negative test outcome is interpreted to mean, an accordance with the respective embodiment: Unfavorable and/or insufficient immune system homeostasis and/or intestinal immune homeostasis, and/or bad intestinal health. Further, the negative test outcome may mean: insufficient gut colonization status, mucosal immune barrier function, intestinal immune system status, and intestinal adaptive immune system status, the presence of an aberrant microbiota, dysbiosis, archaea-deficiency, and M. smithii deficiency, and/or an increased risk for suffering and/or developing one or more selected from the groups consisting of colic, allergy, asthma, IBD, colon cancer and diarrhoea.

In preferred embodiments, the present invention also provides methods of administrating M. smithii.

M. smithii is preferably administered for treating and/or preventing a condition and/or disorder as specified in the present specification. In some embodiments, M. smithii is administered for addressing archaea-deficiency, including but not necessarily limited to a M. smithii deficiency in an individual.

In some embodiments, M. smithii is administered not necessarily or not only for treating and/or preventing a disorder, but for promoting and/or improving intestinal health in general.

In some embodiments, M. smithii is administered for promoting and/or improving immune system homeostasis, intestinal immune homeostasis, intestinal health, gut colonization status, mucosal immune barrier function, intestinal immune system status, intestinal adaptive immune system status.

In an embodiment, M. smithii is administered for regulating immune status and/or homeostasis, for example in infants. In an embodiment, inactivated M. smithii is administered for modulating one or more selected from immune status, immune homeostasis, for example IgA homeostasis and T cell homeostasis, in newborns, infants and toddlers.

In an embodiment, M. smithii is administered for treating and/or preventing: an aberrant microbiota, dysbiosis, archaea-deficiency, and M. smithii deficiency.

In an embodiment, M. smithii is administered for treating and/or preventing one or more selected from the group consisting of: colic, allergy, asthma, colon cancer, IBD and diarrhoea.

In an embodiment, the M. smithii is administered, in the form of an appropriate formulation, to an individual, for example a human, in need thereof.

In an embodiment, the M. smithii is administered to an individual early in life. In an embodiment, M. smithii is administered to one or more selected from: newborns, infants, and toddlers up to 6, 5 or 4 years.

A newborn is a human individual having an age of up to 28 days. For the purpose of the present specification, an infant is a human individual having an age of more than 28 days and up to one (1) year. A toddler is a human individual having an age of more than one year and up to three (3) years. For the purpose of the present specification, infants and children having an age up to 6 years, preferably up to 5 years and most preferably up to 4 years represent the main subject of the present invention. Therefore, the term “toddler” is generally used in this specification to include children up to the above indicated age.

It is, however, noted that human individuals of 50 years and older are also a subject of the present invention. For example, the invention provides assessing and/or determining M. smithii in these individuals for determining a risk of developing colon cancer, and the use of M. smithii, live and/or inactivated, for the prophylaxis and/or treatment of colon cancer in these individuals.

In an embodiment, M. smithii is used for regulating microbiota and/or immune system development in early life, in particular in human individuals selected from: newborns, infants, and toddlers up to 6, 5, or 4 years.

M. smithii is preferably administered in the form of a formulation and/or composition that comprises M. smithii. In the formulation, M. smithii may be live or viable. Alternatively, inactivated M. smithii may be administered. In an embodiment, the formulation comprises live and inactivated M. smithii.

The formulation preferably comprises M. smithii and at least one pharmaceutically acceptable carrier. In an embodiment, the formulation is provided in the form of a in tablet, pill, capsule, formula milk, nutritional composition, powder form in sachet, or in the form of liquid drops. The nutritional composition may be powdered or liquid, for example ready to drink.

M. smithii, or preferably the formulation comprising the same, is preferably administered by enteral, sublingual, buccal, oral, rectal, intrarectal, and intranasal administration. Preferably, the formulation, in particular the one or more carriers, are adapted to the appropriate administration route. It is noted that the administration route will also determine the dosage and/or frequency of administration of M. smithii.

In case of the administration of live M. smithii, the dosage is preferably determined in terms of viable cells, which may be expressed as a number per administration unit and/or per gram of a carrier material. In the present specification, the terms “live M. smithii” and “viable M. smithii” are used interchangeably and refer to M. smithii cells which, when exposed to appropriate conditions, can grow and multiply.

In an embodiment, the formulation provided for administration comprises from 10⁷ to 10¹², preferably 10⁸ to 10¹¹ viable cells per kg body weight of recipient. In the case of a toddler receiving viable M. smithii, the formulation may directly comprise 5×10⁷ to 30×10¹² viable cells per unit of administration, preferably 5×10⁸ to 30×10¹¹ viable cells, for example

The number of viable M. smithii per gram of formulation is preferably determined by way of staining protocols that have been reported in the literature, in particular live/dead staining protocols, as disclosed in WO 2020/002543 A1.

The dosage preferably depends on factors that need to be assessed when the administration is prescribed or recommended, such as the frequency of administration, the delivery vehicle (administration form and/or route), and the health status of individual.

M. smithii can be provided by known processes. For obtaining a high amount or ratio of live M. smithii, freeze-drying is a preferred method. Freeze drying may result up to 40% viable cells when the freeze dry process is optimized. Alternatively, spray-drying protocols are also feasible for obtaining M. smithii, including live M. smithii. For producing inactivated M. smithii, inactivation protocols may be used, for example as disclosed in WO 2020/002543 A1.

Table 2 below concerns particular embodiments of the invention. Experimental examples for illustrating the invention are disclosed further below.

TABLE 2
Amino acid sequences for practicing embodiments of the claimed invention

| SEQ ID | Brief Description | Amino acid (aa) sequence |
|---|---|---|
| 1 | A5UJP0_METS3 | MELPIAPVGRILKNAGAQRVSDDAKIALTEAIEECGNEIA |
| | Marker prot M. smithii | QKAVGFARHA |
| 2 | A5UP44_METS3 | MSEDHNKKEHKHCPFCGHHVDEDDMICPHCGLFIP |
| | Marker prot M. smithii | |
| 3 | A5UNQ8_METS3 | MYRCPRCGTEVDHKSYMENKCPKCRYRILFKNVPEVTR |
| | Marker prot M. smithii | IIKAR |
| 4 | A5UN59_METS3 | MIPIRCLSCGKPVSAYFDEYNKRLAAGEKSKDILDDLGL |
| | Marker prot M. smithii | NRYCCRRMLISHVETWE |
| 5 | A5UMN7_METS3 | MSEIPKAPIARIIKDTGAERVSEDAKAELAEYLEEVARDV |
| | Marker prot M. smithii | AIEANNVAKIAKRKTIKPEDIKLAIKNLE |
| 6 | MTD_METS3 | MVVKIGIIKSGNIGTSPVLDLLLDERADRPNIDVRVFGSG |
| | Marker prot M. smithii | AKMNPEQVEDVVPKVDQFDPDFCIFISPNPGAPGPAKAR |
| | | ELLSEKDIPAIIIGDAPGKGKKDEMDEQGLGYIIVMSDPM |
| | | IGAKREWLDPTEMAIFNADILKVLAETGALRLVQNTIDG |
| | | VIDGAAAGNIELPKLIITAEKAVEAAGFENPYAKAKAIA |
| | | AYEMAGAVANLDMKGCFMTKGFENFIPLVAAAHEMA |
| | | ASAAALADEAREIEKGNDSVLRTPHMKEGNTGCKTDLI |
| | | SKPE |
| 7 | A5ULN2_METS3 | MQIYNDKIDLYGQDGKLLKSEVSLDAISPLKNPAIAKMI |
| | Marker prot M. | FDIKRSCAVNLASIEKGLRTGAMGGKSVFIPGRELDLPIV |
| | smithii | ENAELIADKIKRIVQVNEDDATNVSLINNGQQLLIQLPNE |
| | | RLRVAADYSIASLLSGSATIQSIIDTFDINMFDASTIKTAV |
| | | MGAYPQTVDLAGANISALLGPPVLLEGLGYGLRNVMA |
| | | NHVVAITNKNTLNAAALSSIMEQTAMFETGDASGAFER |
| | | MHLLTLAYQGLNADNLVFDLVKENVKGTVGTVIQSLV |
| | | GRAIEDNVIKASRKMNSGFINYEPVDWALWNAYAAAG |
| | | LLAAVIVNVGAARAAQAVASTVLYYNDILEYETGLPSV |
| | | DFGRTMGVGVGFSFFSHSIYGGGGPGTFHGNHVVTRHS |
| | | KGCAIPCASAAMCLDAGTQMFSVQRTSSLIGTVYGTIDN |
| | | LRNPIINVAKSAGELKI |
| 8 | A5ULZ6_METS3 | MAKFDDKVDLYDDRGSLVVSDVPIEALSPLRNTAIQNIV |
| | | KGVKRTVAVNLEGLEKSVKTGSVGGDKSKILGRELDIDI |
| | | VANAGAIAEKMKEMIQISEDDDTKVEPISGGKRLLVQVP |
| | | SKRIDVAAEYSVAPLSTATSLVQAIIDVCDVSIYDANFVK |
| | | AAVLGRCPQSVDYKGSNIATMLDIPQKLEGAGYALRGV |
| | | KANDFAAATLKNTFQATALASIFEQTAMFEMGDAIGAY |
| | | ERLHLLGLAYQGMNADNMVLDLVKDNAKEGTVGSVV |
| | | NGTIARAEADGVIAPQKDLTDFSIYNTDDAALWNAYAA |
| | | AGAAAAVMVNIGAARAAQGIPSTLLYFNDNIEFATGLPS |
| | | IDYGRAEGVAVGFSFFSHSIYGGGGPGLFNGNHVVTRHS |
| | | KGFCIPCVAAAMSLDAGTQLFSPEATSGLIKEVYSQVDE |
| | | FREPLKYVALAADDIKGDI |
| 9 | mAb1 VH | EVQLVQSGAEVKKPGESLKISCKGSGYSFTSYWIGWVR |
| | | QMPGKGLEWMGIIYPGDSDTRYSPSFQGQVTISADKSIS |
| | | TAYLQWSSLKASDTAMYYCARAGFRGYCSGGSCYRLP |
| | | YWGQGTLVTVSS |
| 10 | mAb1 VL | QSVLTQPPSVSAAPGQKVTISCSGSSSNIGNNYVSWYQQ |
| | | LPGTAPKLLIYDNNKRPSGIPDRFSGSKSGTSATLGITGL |
| | | QTGDEADYYCGTWDVLAARSVFGGGTKLTVLG |
| 11 | mAb2 VH | EVQLVQSGAEVKKPGESLKISCKGSGYSFTSYWIGWVR |
| | | QMPGKGLEWMGIIYPGDSDTRYSPSFQGQVTISADKSIS |
| | | TAYLQWSSLKASDTAMYYCARLATGPHGIASAGRRYL |
| | | DYWGQGTLVTVSS |
| 12 | mAb2 VL | QSVLTQPPSVSAAPGQKVTISCSGSSSNIGNNYVSWYQQ |
| | | LPGTAPKLLIYDNNKRPSGIPDRFSGSKSGTSATLGITGL |
| | | QTGDEADYYCGTWDSRGFGVFGGGTKLTVLG |
| 13 | mAb3 VH | QVQLVQSGAEVKKPGASVKVSCKASGYTFTSYGISWVR |
| | | QAPGQGLEWMGWISAYNGNTNYAQKLQGRVTMTTDT |
| | | STSTAYMELRSLRSDDTAVYYCARGWGDWEMDYWGQ |
| | | GTLVTVSS |
| 14 | mAb3 VL | QSVLTQPPSVSAAPGQKVTISCSGSSSNIGNNYVSWYQQ |
| | | LPGTAPXLLIYDNNKRPSGIPDRFSGSKSGTSATLGITGL |
| | | QTGDEADYYCRTYDWTHSRWVFGGGTKLTVLG |
| 15 | mAb4 VH | EVQLVESGGGLVQPGGSLRLSCAASGFTFSSYSMNWVR |
| | | QAPGKGLEWVSYISSSSSTIYYADSVKGRFTISRDNAKN |
| | | SLYLQMNSLRAEDTAVYYCARDFPNTEAFDIWGQGTLV |
| | | TVSS |
| 16 | mAb4 VL | QSALTQPASVSGSPGQSITISCTGTSSDVGGYNYVSWYQ |
| | | QHPGKAPKLMIYEVSNRPSGVSNRFSGSKSGNTASLTIS |
| | | GLQAEDEADYYCSSYDVFGGGTKLTVLG |
| 17 | mAb5 VH | EVQLVQSGAEVKKPGESLKISCKGSGYSFTSYWIGWVR |
| | | QMPGKGLEWMGIIYPGDSDTRYSPSFQGQVTISADKSIS |
| | | TAYLQWSSLKASDTAMYYCARAKVGQRYSSGRYFFDY |
| | | WGQGTLVTVSS |
| 18 | mAb5 VL | EIVLTQSPATLSLSPGERATLSCRASQSVSSYLAWYQQK |
| | | PGQAPRLLIYDASNRATGIPARFSGSGSGTDFTLTISSLEP |
| | | EDFAVYYCQQGAKDNPMTFGQGTKVEIKR |
| 19 | Linker scFV | GGGGSGGGGSGGGGST |
| 20 | mAb1 H-CDR1 | GYSFTSYWIG |
| 21 | mAb1 H-CDR2 | IYPGDSDT |
| 22 | mAb1 H-CDR3 | ARAGFRGYCSGGSCYRLPY |
| 23 | mAb1 L-CDR1 | SSNIGNNY |
| 24 | mAb1 L-CDR2 | DNN |
| 25 | mAb1 L-CDR3 | GTWDVLAARSV |
| 26 | mAb2 H-CDR1 | GYSFTSYWIG |
| 27 | mAb2 H-CDR2 | IYPGDSDT |
| 28 | mAb2 H-CDR3 | ARLATGPHGIASAGRRYLDY |
| 29 | mAb2 L-CDR1 | SSNIGNNY |
| 30 | mAb2 L-CDR2 | DNN |
| 31 | mAb2 L-CDR3 | GTWDSRGFGV |
| 32 | mAb3 H-CDR1 | GYTFTSYGIS |
| 33 | mAb3 H-CDR2 | ISAYNGNT |
| 34 | mAb3 H-CDR3 | ARGWGDWEMDY |
| 35 | mAb3 L-CDR1 | SSNIGNNY |
| 36 | mAb3 L-CDR2 | DNN |
| 37 | mAb3 L-CDR3 | RTYDWTHSRWV |
| 38 | mAb4 H-CDR1 | GFTFSSYSMN |
| 39 | mAb4 H-CDR2 | ISSSSSTI |
| 40 | mAb4 H-CDR3 | ARDFPNTEAFDI |
| 41 | mAb4 L-CDR1 | SSDVGGYNY |
| 42 | mAb4 L-CDR2 | EVS |
| 43 | mAb4 L-CDR3 | SSYDV |
| 44 | mAb5 H-CDR1 | GYSFTSYWIG |
| 45 | mAb5 H-CDR2 | IYPGDSDT |
| 46 | mAb5 H-CDR3 | ARAKVGQRYSSGRYFFDY |
| 47 | mAb5 L-CDR1 | QSVSSY |
| 48 | mAb5 L-CDR2 | DAS |
| 49 | mAb5 L-CDR3 | QQGAKDNPMT |

A percentage of “sequence identity” may be determined by comparing the two sequences, optimally aligned over a comparison window, wherein the portion of the polypeptide sequence in the comparison window may comprise additions or deletions (i.e. gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison is conducted by global pairwise alignment, e.g. using the algorithm of Needleman and Wunsch (1970) J. Mol. Biol. 48:443. The percentage of sequence identity can be readily determined for instance using the program Needle, with the BLOSUM62 matrix, and the following parameters gap-open=10, gap-extend=0.5.

In an embodiment, any amino acid residue replacement and/or substitution in a sequence having a certain sequence identity with any one of the sequences disclosed in this specification is preferably under the proviso that any amino acid that is non-identical from the amino acid at the corresponding position in the specified sequence is conservatively substituted, in accordance with substitutions as specified in Table 3 below showing amino acid residues that are substitutable among each other:

TABLE 3
Conservative amino acid substitutions

| | Group of substitutable |
|---|---|
| Substitution type | amino acid residues |
| Aliphatic residues | I, L, V, and M |
| Cycloalkenyl-associated residues | F, H, W, and Y |
| Hydrophobic residues | A, C, F, G, H, I, L, |
| | M, R, T, V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, |
| | N, Q, R, S, P, and T |
| Flexible residues | Q, T, K, S, G, P, D, E, and R |
| valine-leucine-isoleucine | V, L and I |
| phenylalanine-tyrosine | F and Y |
| lysine-arginine | K and R |
| alanine-valine | A and V |
| asparagine-glutamine | N and Q |

While certain of the preferred embodiments of the present invention have been described and specifically exemplified above, it is not intended that the invention be limited to such embodiments. Various modifications may be made thereto without departing from the scope and spirit of the present invention, as set forth in the following claims. Herein below, examples of the invention are disclosed. These examples are for illustration only and are not intended to limit the scope of the present invention.

### EXAMPLES

### Example 1: M. smithii in Infancy and Early Childhood

### Introduction

It is not known whether M. smithii colonizes the intestine in coordination with the immune system in an age-dependent manner; and, moreover, whether timely M. smithii colonization correlates with normal immune system development.

Mucosal antibodies are perpetually secreted in the intestine, called immunoglobulin-A (sIgA), which coat the surface of symbiotic gut bacteria, a process that helps foster immune tolerance and microbiota diversity in the host.

The secretion and quality of sIgA is regulated by the microbiota composition, and, in turn, sIgA coating of the microbiota helps to foster tolerance and mutualistic interactions. (Sutherland D B, Suzuki K, Fagarasan S, Fostering of advanced mutualism with gut microbiota by Immunoglobulin A, 2016, Immunological Reviews; Sutherland D B, Fagarasan S, IgA synthesis: a form of functional immune adaptation extending beyond gut, 2012, Current Opinion in Immunology).

Microbial species that preferentially drive adaptive sIgA secretion and microbiota coating in early-life are of great interest to achieving host-microbiota mutualism and immune system homeostasis.

Poor sIgA coating is associated with aberrant microbiota, dysbiosis and inflammation. Thus, there is a need to identify effective biomarkers to monitor aberrant microbiota and immune system development.

Correlates between M. smithii colonization in early-life and sIgA coating of microbiota are evaluated here. The first goal is to understand whether M. smithii colonization status can potentially be used as a biomarker to evaluate normal versus aberrant microbiota and poor immune system development in infants.

### Methods

Ethical approval is obtained in accordance with the standard CER-VD of Vaud, Switzerland. A healthy cohort of 30 individuals of a Kindergarden in Lausanne agreed to participate in the study. Participants were grouped in three age groups (n=10 per group): 6-12 months, 12-18 months, and 18-24 months. Stools were collected from the participants.

1 gram of stool was collected and suspended in 1 mL of 4% PFA in PBS for 10 minutes. Suspensions were vigorously mixed and diluted with 10 mL PBS. Suspensions were centrifuged at 1000×g for 10 minutes and the supernatant collected. The number of cells per mL were counted and cell densities adjusted to 10⁷ per mL. Invitrogen goat anti-human IgA Fc Secondary antibody, FITC (Catalog H14101) staining protocol was performed. Flow cytometer analysis was performed using a BD LSRFortessa Cell Analyzer. The M. smithii biomarker F420 was excited by 405 nm laser (the purity of the M. smithii gated population was confirmed by cell sorting F420+ gated events with BD FACSAria II and PCR tests). The percentage of F420+ events and percentage IgA+ coated bacteria cells were analyzed by flow-jo software and plotted using excel software.

### Results

Infant stool samples were harvested from healthy infants in Lausanne Switzerland with respective groups aged 6-12 months, 12-18 months and 18-24 months.

Microbial cell populations extracted from stool samples were analyzed by flow cytometer using two key parameters: i) % IgA coated microbial cells ii) % F420+405 nm excited cells.

F420 is a co-enzyme produced exclusively by M. smithii in the human microbiota that is excited at 405 nm fluorescence. This is confirmed by cell sorting and PCR identification and additionally by fluorescent microscopic analysis.

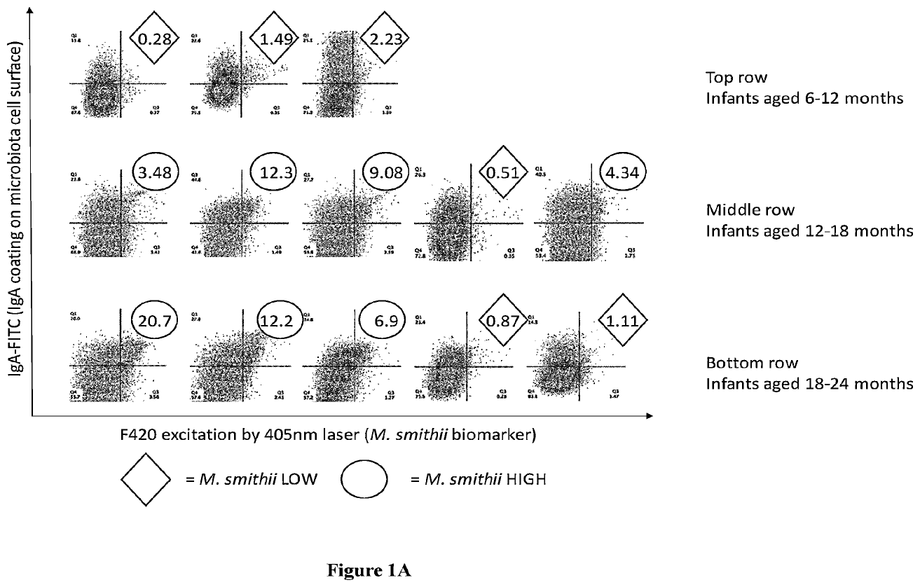

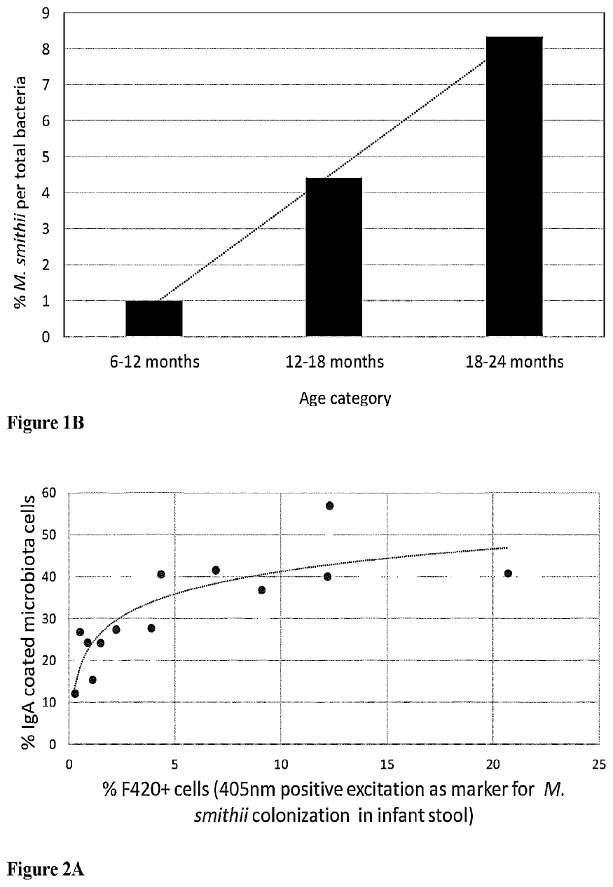

The data show absence or low levels of M. smithii biomarker in infants aged 6-12 months, as shown in FIGS. 1A and 1B. A burst of the M. smithii biomarker is observed in the 12-18 month age group with a marked increase of the M. smithii biomarker observed in the 18-24 months aged infant group.

The 70% of the subjects aged 12-24 months showed clear elevation of M. smithii biomarker labeled “M. smithii high” versus the 6-12 months age group that were labeled “M. smithii low”.

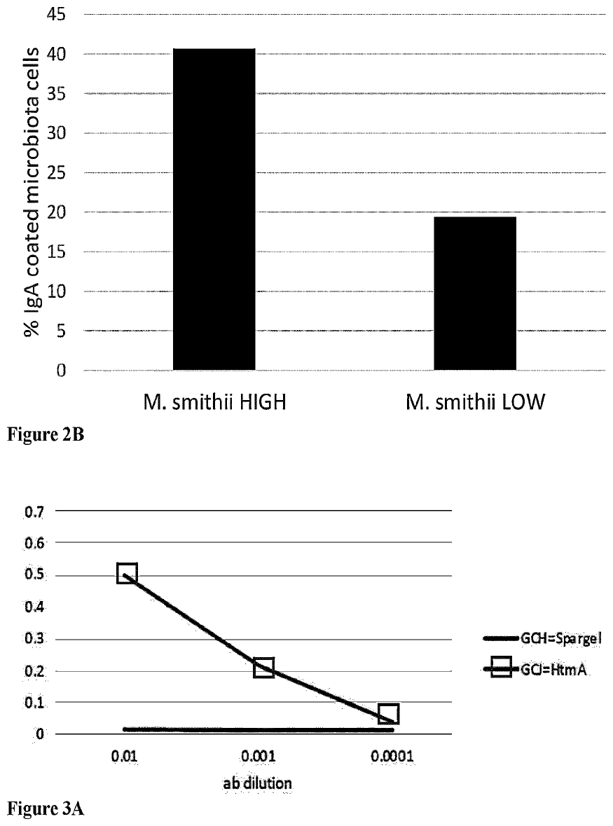

Increase of the flow cytometer M. smithii biomarker correlated positively with level of IgA coated microbiota cells, as shown in FIG. 2A. FIG. 2B shows that Infants in the 18-24 months group that were “M. smithii high” had double the percentage of IgA coated microbiota cells with on average 40.7% IgA coated microbiota cells versus “M. smithii low” infants from the same age group having only 19.4% IgA coated microbiota cells.

### Discussion and Conclusion

The data show a correlation between infant age and the percentage of M. smithii biomarker monitored in stool samples. The absence of M. smithii in tested infants is associated with lower levels of sIgA microbiota coating. The data reveals that the M. smithii levels in stool samples corresponds directly with sIgA coating level and therefore provide information about the intestinal immune system status during early-life. Clinical studies suggest intestinal archaea are inversely correlated with asthma (Barnett et al, Intestinal archaea inversely associated with asthma, 2019, Journal of Allergy and Clinical Immunology) raising the possibility of either indirect or direct interactions between M. smithii with immunological function. Our study shows the first relationship between M. smithii with mucosal immune phenotype in infants.

IgA coating is a T cell dependent process (Sutherland D B, Suzuki K, Fagarasan S, Fostering of advanced mutualism with gut microbiota by Immunoglobulin A, 2016, Immunological Reviews) suggesting a coordination between the colonization of M. smithii with T cell differentiation and immune system phenotype. T cells are master regulators of the adaptive immune system that underpin immunological homeostasis and human health. Further studies in gnotobiotic mice inoculated with M. smithii and a minimal bacterial consortium will enable the elucidation of cellular pathways involved.

Surprisingly, M. smithii expansion in infant microbiota is shown to be age-dependent and coordinated with immune system development. The study demonstrates that M. smithii can be used as a biomarker to infer intestinal immune system status in infants.

### Example 2: Selection of Markers for Identifying M. smithii

For developing a rapid test that allows monitoring M. smithii, the proteome of M. smithii (strain ATCC 35061; DSM 861; OCM 144; PS) was retrieved and evaluated.

Eight candidate marker proteins were identified, shown in Table 2. Several markers were selected based on the presence of strong and characteristic 3.5 kD, 4.3 kD and 5.6 kD peaks appearing in mass spectrometry analysis. For these peaks, the RNA polymerase subunit is assumed to be a likely match, as it is highly expressed.

The histone HmtA (SEQ ID NO: 1), a histone having 65 amino acids and a MW of about 6.9 kDA (SEQ ID NO: 1) was selected for further evaluation as a marker that is specific to M. smithii. HmtA has a homo-dimer structure having 71.88% sequence similarity with Histone HMFA of Methanothermus fervidus.

### Example 3: Preparation of Antibodies Specific for HMTA

Five monoclonal antibodies mAb1-mAb5 were selected from a semi-synthetic naive library of human origin, the Tomlinson I,J library, which was kindly provided by MRC, Cambridge, UK. This library contains approximately 109 independent scFv recombinant antibodies inserted in the pIT2 vector (de Wildt et al., 2000). GST fusion proteins used to select recombinant antibodies were prepared according to the procedure disclosed in C. Blanc et al. “Use of In Vivo Biotinylated GST Fusion Proteins to Select Recombinent Antibodies”, ALTEX. 2014, vol. 31, no. 1, p. 37-42.

The scFv (Single chain variable fragments) recombinant antibodies were prepared according to the general structure N-VH-VL-C (scFV) were prepared using a linker shown in Table 2 (SEQ ID NO.: 19) between VH and VL regions.

The amino acid sequences of the VH and VL sequences as well as of the linker are shown in Table 2.

### Example 4: ELISA Test

The binding properties of all antibodies were tested by ELISA. A glutathione S-transferase (GST)-HmtA chimeric construct was prepared and further biotinylated in vivo by bacteria at the N-terminus (GST) end of the HmtA construct (GCJ construct). The construct was immobilized in coated polystyrene plates. A control chimeric GST construct (GCH) lacking HmtA was prepared, biotinylated and immobilized analogously.

HmtA and control constructs were exposed to each of the five scFVs of Example 3 under binding conditions.

Binding was revealed by a secondary HRP polyclonal antibody specific to the (GST)-HmtA chimeric constructs, and the colorimetric reaction was measured.

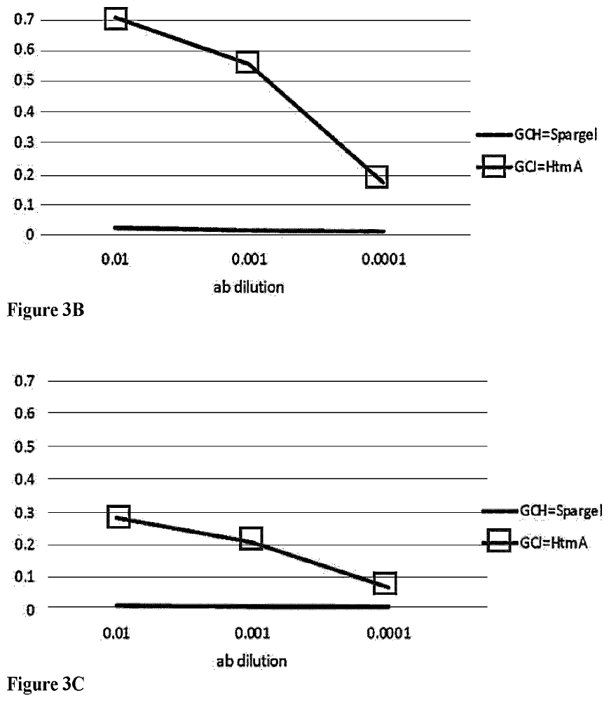

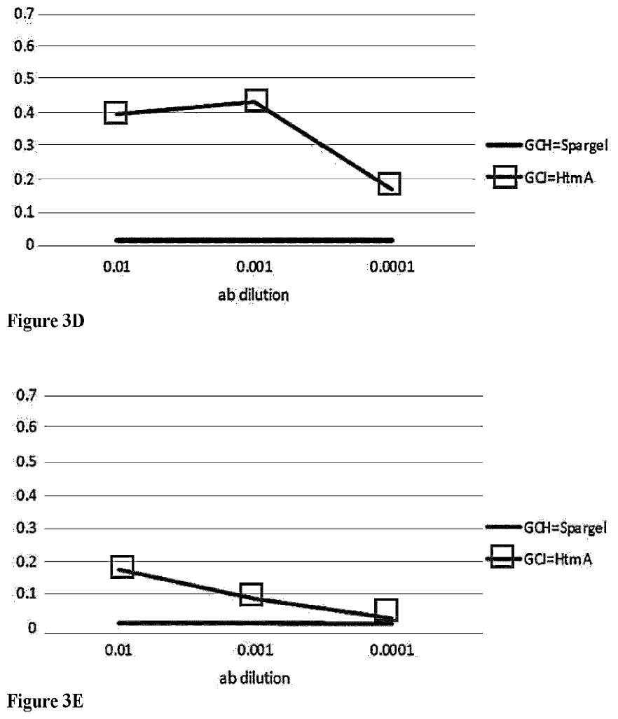

The results are shown in FIGS. 3A-3E for each of the scFVs. All scFVs bind to the HmtA containing construct but not to the control construct, with the colorimetric being dependent on the concentration of the antibodies as shown in the figures.

### Example 5: Detection of M. smithii in Gnobiotic Mice

We compare microbiota samples that contain M. smithii (HmtA positive) versus microbiota samples that do not contain M. smithii (HmtA negative) using gnotobiotic mice that are colonized with M. smithii plus 12 defined commensal bacteria species.

As a control, gnotobiotic mice that contain only the 12 defined commensal bacteria species but without any M. smithii. The antibodies of the invention give positive measurements for stool samples taken from M. smithii positive mice.

### Example 6: Discrimination Between M. smithii and Methanobrevibacter boviskoreani

M. boviskoreani is an archaea species dominant in adult pigs, which is related to M. smithii. Samples containing M. boviskoreani that do not contain M. smithii are obtained. The HmtA antibodies work only in samples containing the M. smithii archaea species, but no binding is detected in samples containing only M. boviskoreani.

### Example 7: Discrimination Between M. smithii and M. stadtmonae

M. stadtmonae is cultured as a monoculture. The anti-HmtA antibodies of the invention do not detect this alternative archaea species.

### Example 8: Archaea-Deficiency Treatment by Oral M. smithii Administration

### Objective and Background

The goal of this example is to find out if orally administered archaea can treat archaea-deficiency. Specifically, gnotobiotic mice with defined microbiome composition that are archaea-deficient are treated by administration of M. smithii archaea orally to determine intestinal archaea colonization status of treated mice.

Infants lacking archaea colonization in early life due to factors that may include mode of birth, lack of human milk, malnutrition, are missing the keystone species M. smithii that functions as the primary hydrogen sink of fermenting bacteria in the human gut. It is not known whether administering M. smithii archaea orally can stimulate archaea colonization and treat archaea-deficiency. It is not known if orally administered archaea can efficiently anchor and establish itself in the intestinal mucosa which is a requirement for stable colonization and integration to the gut microbiome. Early life (ages 0-4) provides a window of opportunity to establish a diversified microbiome which is linked to development of the immune system. Experimental mouse models are a gold-standard in preclinical screening enabling to evaluate the therapeutic efficacy of M. smithii for treatment of archaea-deficiency in humans. Gnotobiotic mice called Oligo MM12 maintained in germ-free facilities, having a defined and controlled microbiome have been described and provide an experimental model to evaluate treatments for archaea-deficiency.

Oligo MM12 mice were first defined by Brugiroux, S., et al. (2016) Genome-guided design of a defined mouse microbiota that confers colonization resistance against Salmonella enterica serovar Typhimurium. Nature Microbiology 2:16215. doi: 10.1038/nmicrobiol.2016.215

### Methods and Results

C57BL/6 Oligo MM12 gnotobiotic mice were established devoid of archaea and colonized with a defined consortium of bacteria consisting of: Akkermansia muciniphilia YL44, Bacteroides caecimuris 148, Muribaculum intestinale YL27, Turicimonas muris YL45, Bifidobacterium longum subsp. animalis YL2, Enterococcus faecalis KB1, Acutalibacter muris KB18, Clostridium clostridioforme YL32, Blautia coccoides YL58, Flavonifractor plautii YL31, Lactobacillus reuteri 149, Clostridium innocuum 146. The experimental mice were maintained in germ-free facilities.

6-week-old archaea-deficient C57BL/6 OligoMM12 mice (n=5 males, n=3 females) were treated with a single dose of 109 lyophilized viable M. smithii archaea organisms administered orally. Successful M. smithii colonization of the animals was confirmed by screening fecal samples at weekly intervals for 8 weeks via PCR, FACS and/or 16s rRNA sequencing (Oxford nanopore). All three methods confirmed the colonization with M. smithii. Two breeding pairs were set up. The resulting offspring were again tested at 4, 6 and 8 weeks of age to confirm successful vertical transfer of M. smithii.

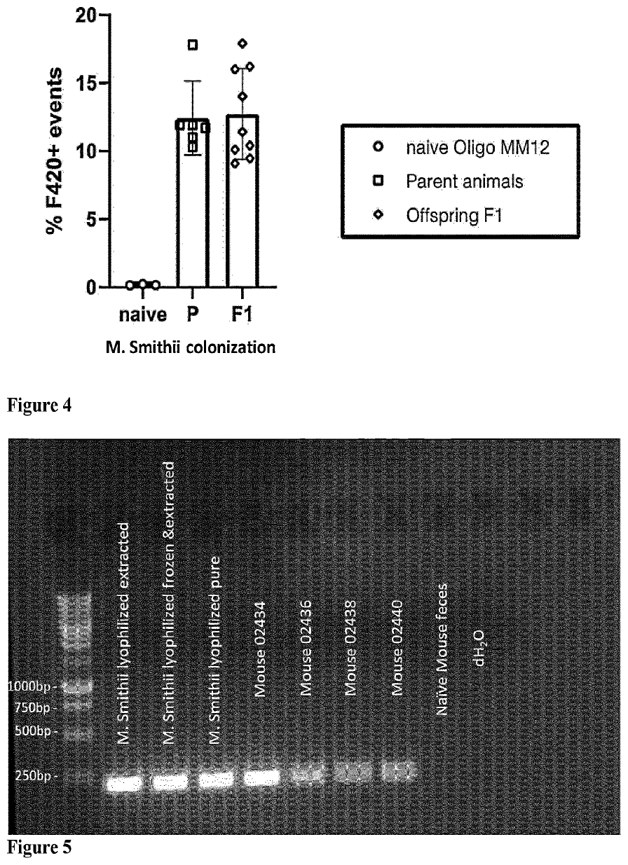

FIG. 4 shows M. smithii load in feces. Feces of naïve Oligo MM12 mice as well as adult mice inoculated with a single dose of M. smithii and of the offspring thereof were washed and analyzed by flow cytometry. Depicted are F420⁺ events (biomarker of M. smithii) as percent of parent gate (i.e. total single bacteria). Data shown were obtained from two independent experiments. Parent and offspring mice are stably colonized with on average 12% relative abundance of M. smithii in their microbiome while naïve control mice have 0% relative abundance M. smithii in their microbiome.

FIG. 5 shows M. smithii colonization confirmed by fecal PCR test. The 222 bp PCR product from M. smithii nifH gene is amplified in 30 cycles with the primers shown below, following a method adapted from Ufnar, J. A., et al. (2006). Detection of the nifH gene of Methanobrevibacter smithii: a potential tool to identify sewage pollution in recreational waters. Journal of applied microbiology, 101 (1), 44-52. forward AACAGAAAACCCAGTGAAGAG-3′ (SEQ ID NO: 50) reverse AGTAAAGGCACTGAAAAACC-3′ (SEQ ID NO: 51)

In FIG. 5, rows show 1-3 positive control for M. smithii, rows 4-7 are representative of M. smithii colonized Oligo MM12 mouse fecal samples, row 8 is a archaea-deficient Oligo MM12 mouse fecal sample, and row 9 is the negative control (H₂0).

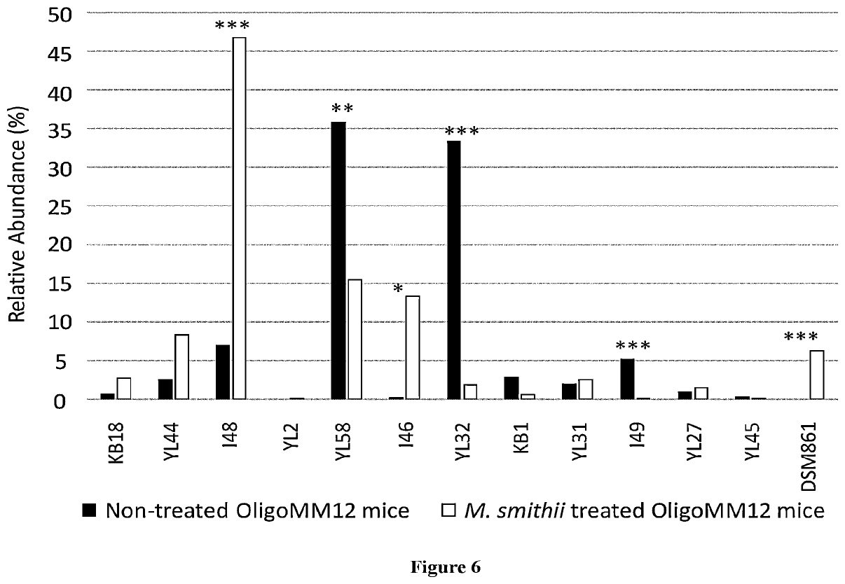

Regarding FIG. 6, fecal samples were collected from OligoMM12 mice either non-treated (n=8) or treated with M. smithii (n=12) and 16S rRNA sequencing Oxford Nanopore qPCR was performed to determine the microbiota composition as mean percentage relative abundance. Treatment with M. smithii led to a highly significant increase in Bacteroides caecimuris (148) and significant increase in Clostridium innocuum (146) compared to non-treated mice. While there was a marked reduction in Enterocloster clostridioformis (YL32) in M. smithii (DSM861) treated mice versus non-treated mice. Statistical T-test were calculated by excel software (***P-value<0.001 and **P-value<0.01 and *P-value<0.05).

TABLE 4
Mean relative abundance of fecal microbiome composition
from OligoMM12 mice treated with M. smithii (n =
12) versus non-treated (n = 8).

| | Non-treated | M. smithii |
|---|---|---|
| | colony | treated colony |
| | (% relative | (% relative |
| Species | abundance) | abundance) |
| Acutalibacter muris KB18 | 0.7 ± 0.3 | 2.7 ± 1.9 |
| Akkermansia muciniphila YL44 | 2.7 ± 1.9 | 8.3 ± 13.5 |
| Bacteroides caecimuris I48 | 7.6 ± 2.9 | 46.9 ± 17.8 |
| Bifidobacterium animalis YL2 | 0 | 0 |
| Blautia coccoides YL58 | 39.7 ± 11.3 | 15.5 ± 6.4 |
| Clostridium innocuum I46 | 0.2 ± 0.2 | 13.3 ± 12.8 |
| Enterocloster clostridioformis YL32 | 36.7 ± 12 | 1.8 ± 1.6 |
| Enterococcus faecalis KB1 | 3.1 ± 12.8 | 0.6 ± 0 |
| Flavonifractor plautii YL31 | 2.2 ± 0.8 | 2.5 ± 0.75 |
| Limosilactobacillus reuteri I49 | 5.7 ± 1.4 | 0.02 ± 0.33 |
| Muribaculum intestinale YL27 | 1.1 ± 0.46 | 1.4 ± 1.4 |
| Turicimonas muris YL45 | 0.3 ± 0.15 | 0.1 ± 0.13 |
| Methanobrevibacter smithii DSM861 | 0 | 6.2 ± 3 |

### CONCLUSIONS

Orally administering lyophilized M. smithii was demonstrated to treat archaea-deficiency enabling stable archaea colonization. The data demonstrate that infants with stunted archaea colonization in early life can be effectively treated by orally administering M. smithii to enable archaea colonization within the normal time window (age 0-4) when the immune system is developing.

qPCR data indicate that archaea-treatment significantly shifts the microbiome composition (FIG. 6, Table 4). Archaea treatment enables to modulate the developing microbiome in favor of archaea-associated microbiome that support fiber fermentation, production of short chain fatty acids and normal development of the immune system including T cell homeostasis and IgA regulation. Notably, the species Bacteroides caecimuris (148) and Clostridium innocuum (146), associated with carbohydrate and polysaccharide metabolism, were significantly enhanced in OligoMM12 mice treated with M. smithii. The interplay between microbiome and immune system in early life fosters immune system homeostasis and microbiome resilience (more resistant to dysbiosis) shown elsewhere to have lasting impact into adulthood.

1.-21. (canceled)

22. A method for monitoring and/or assessing one or more selected from the group consisting of: immune system homeostasis, intestinal immune homeostasis, and/or intestinal health, in a human individual selected from the group consisting of: a newborn, infant or a toddler up to 4 year, the method comprising:
determining whether M. smithii is present in a sample collected from the human; and if so, determining or estimating a quantity of M. smithii in the sample, and,
finding insufficient immune system homeostasis, intestinal immune homeostasis, and/or intestinal health if M. smithii is absent or is present at a quantity that is below a threshold level.

23. The method of claim 22, wherein said intestinal immune homeostasis and/or intestinal health includes one or more selected from the group consisting of: gut colonization status, mucosal immune barrier function, intestinal immune system status, and intestinal adaptive immune system status.

24. The method of claim 22, which comprises providing a binding molecule that specifically binds to a marker of M. smithii.

25. The method of claim 24, wherein determining whether M. smithii is present in a sample collected from the human; and if so, determining or estimating a quantity of M. smithii in the sample comprises detecting binding of said binding molecule to said marker.

26. The method of claim 24, wherein said marker is selected from the proteins comprising an amino acid sequence of any one of SEQ ID NO: 1-8, preferably said marker protein is HmtA comprising amino acid sequence SEQ ID NO: 1.

27. The method of claim 24, wherein said binding molecule is selected from the group consisting of:
an oligo- or polynucleotide, preferably a probe; and
a binding protein, preferably an immunoglobulin superfamily (IgSF) protein or fragment thereof, preferably an antibody or fragment thereof.

28. The method of claim 22, wherein said sample is a stool sample or is prepared from a stool sample.

29. A method for diagnosing one or more selected from the group consisting of: an aberrant microbiota, dysbiosis, archaea-deficiency, and M. smithii deficiency, in a human individual selected from the group consisting of: a newborn, infant or a toddler up to 4 years, the method comprising:
determining whether M. smithii is present in a sample collected from the human; and if so, determining or estimating a quantity of M. Smithii in the sample, and,
diagnosing an aberrant microbiota, dysbiosis, archaea-deficiency, and/or M. smithii deficiency if M. smithii is absent or is present at a quantity that is below a threshold level.

30. The method of claim 29, which comprises providing a binding molecule that specifically binds to a marker of M. smithii.

31. The method of claim 30, wherein determining whether M. smithii is present in a sample collected from the human; and if so, determining or estimating a quantity of M. smithii in the sample, comprises detecting binding of said binding molecule to said marker.

32. The method of claim 30, wherein said marker is selected from the proteins comprising an amino acid sequence of any one of SEQ ID NO: 1-8.

33. The method of claim 30, wherein said binding molecule is selected from the group consisting of:
an oligo- or polynucleotide, preferably a probe; and
a binding protein, preferably an immunoglobulin superfamily (IgSF) protein or fragment thereof, preferably an antibody or fragment thereof.

34. A method for:
treating one or more selected from the group consisting of: an aberrant microbiota, archaea-deficiency, M. smithii deficiency, colic, allergy, asthma, colon cancer, IBD and diarrhoea, and/or,
promoting one or more selected from the group consisting of: immune system homeostasis, intestinal immune homeostasis, intestinal health, gut colonization, mucosal immune barrier function, the intestinal immune system, and the intestinal adaptive immune system, the method comprising, administering to an individual in need thereof, a composition comprising M. smithii.

35. The method of claim 34, wherein said composition comprises live M. smithii.

36. The method of claim 34, wherein the individual is a human individual selected from the group consisting of: newborns, infants, and toddlers up to 6, 5 or 4 years.

37. The method of claim 34, wherein the individual is a human individual of 50 years or older.

38. The method of claim 34, wherein said composition comprises from 10⁷ to 10¹², viable cells per kg body weight of the individual.

39. The method of claim 34, wherein the composition is for oral, rectal, or intrarectal administration.

40. A diagnostic kit for monitoring and/or assessing one or more selected from the group consisting of: immune system homeostasis, intestinal immune homeostasis, and intestinal health, in a human newborn, infant and/or a toddler up to 4 years, the diagnostic kit comprising a binding molecule for detecting and/or determining or estimating a quantity of M. smithii in a sample taken from the individual, wherein said binding molecule is selected from the group consisting of:
an oligo- or polynucleotide, preferably a probe;
a binding protein, preferably an immunoglobulin superfamily (IgSF) protein or fragment thereof, preferably an antibody or fragment thereof.

41. The diagnostic kit of claim 40, wherein said marker molecule is selected from the proteins comprising an amino acid sequence of any one of SEQ ID NO: 1-8.
